(19) United States
(12) Patent Application Publication
(10) Pub. No.: US 2015/0092980 A1
(43) Pub. Date: Apr. 2, 2015

(54) TRACKING PROGRAM AND METHOD

(71) Applicants: Eelke Folmer, Reno, NV (US); George Bebis, Reno, NV (US); Jeff Angermann, Reno, NV (US)

(72) Inventors: Eelke Folmer, Reno, NV (US); George Bebis, Reno, NV (US); Jeff Angermann, Reno, NV (US)

(21) Appl. No.: 14/120,418

(22) Filed: Aug. 23, 2013

(51) Int. Cl. A61B 5/00; A61B 5/11; G06T 7/20

(52) U.S. Cl. CPC A61B 5/4866; G06T 7/2033; A61B 5/1128; A61B 5/1118; G06T 2207/10016; G06T 2207/30196; G06T 2207/30221
USPC 382/103

(57) ABSTRACT

In one embodiment, the present disclosure provides a computer implemented method of determining energy expenditure associated with a user's movement. A plurality of video images of a subject are obtained. From the plurality of video images, a first location is determined of a first joint of the subject at a first time. From the plurality of video images, a second location is determined of the first joint of the subject at a second time. The movement of the first joint of the subject between the first and second location is associated with an energy associated with the movement.

### CROSS REFERENCE TO RELATED APPLICATION

This application claims the benefit of, and incorporated by reference, U.S. Provisional Patent Application Ser. No. 61/692,359, filed Aug. 23, 2012.

### SUMMARY

Certain aspects of the present disclosure are described in the appended claims. There are additional features and advantages of the various embodiments of the present disclosure. They will become evident from the following disclosure.

In this regard, it is to be understood that the claims form a brief summary of the various embodiments described herein. Any given embodiment of the present disclosure need not provide all features noted above, nor must it solve all problems or address all issues in the prior art noted above or elsewhere in this disclosure.

### BRIEF DESCRIPTION OF THE DRAWINGS

Various embodiments are shown and described in connection with the following drawings in which:

FIG. 1 is a photograph of a subject playing an exergame useable in an embodiment of the present disclosure.

FIG. 2 is a visual representation of the sphere and its partitioning into bins for the joint binning process.

FIG. 3: Predicted METs versus ground truth. Figures show predicted METs in one-minute intervals for light (left) and vigorous (right) using three different regression models with each model using the following features: KA uses acceleration; KJB uses joint position; and KA+KJB uses both acceleration and joint position. AA shows predicted MET using the wearable accelerometers. MET's are averaged over 9 subjects. The blue lines show the ground truth MET collected using the portable metabolic system.

FIG. 4 Root mean square (RMS) error of predicted MET versus ground truth for each technique and their averages. Error bars display standard deviation of the RMS error between subjects.

FIG. 5 Recent commercially available depth sensing cameras, such as Microsoft Kinect, allow for accurately tracking skeletal joint positions of a user playing an exergame.

FIG. 6: Kinematic information and EE of a subject playing an exergame is obtained using a portable VO2 metabolic system. From the skeletal joint location data, various motion related features are extracted to train a regression model using the collected ground truth data.

FIG. 7 Based on kinematic information the regression model can then predict the EE of an activity.

### DETAILED DESCRIPTION

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together. Although methods and materials similar or equivalent to those described herein can be used in the practice or testing of the present disclosure, suitable methods and materials are described herein. The disclosed materials, methods, and examples are illustrative only and not intended to be limiting.

Short bouts of high-intensity training can potentially improve fitness levels. Though the durations may be shorter than typical aerobic activities, the benefits can be longer lasting and the improvements to cardiovascular health and weight loss more significant. This observation is particularly interesting in the context of exergames, e.g., video games that use upper and/or lower-body gestures, such as steps, punches, and kicks and which aim to provide their players with an immersive experience to engage them into physical activity and gross motor skill development. Exergames are characterized by short bouts (rounds) of physical activity. As video games are considered powerful motivators for children, exergames could be an important tool in combating the current childhood obesity epidemic.

A problem with the design of exergames is that for game developers it can be difficult to assess the exact amount of energy expenditure a game yields. Heart rate is affected by numerous psychological (e.g., ‘arousal’) as well as physiological/environmental factors (such as core and ambient temperature, hydration status), and for children heart rate monitoring may be a poor proxy for exertion due to developmental considerations. Accelerometer based approaches can have limited usefulness in capturing total body movement, as they typically only selectively measure activity of the body part they are attached to and they can't measure energy expenditure in real time. To accurately predict energy expenditure additional subject specific data is usually required (e.g. age, height, weight). Energy expenditure can be measured more accurately using pulmonary gas (VO2, VCO2) analysis systems, but this method is typically invasive, uncomfortable and expensive.

In a specific example, the present disclosure provides a computer vision based approach for real time estimation of energy expenditure for various physical activities that include upper and lower body movements that is non-intrusive, has low cost and which can estimate energy expenditure in a subject independent manner. Being able to estimate energy expenditure in real time could allow for an exergame to dynamically adapt its gameplay to stimulate the player in larger amounts of physical activity, which achieves greater health benefits.

In a specific implementation, regression models are used to capture the relationship between human motion and energy expenditure. In another implementation, view-invariant, representation schemes of human motion, such as histograms of 3D joints, to develop different features for regression models.

Approaches for energy expenditure estimation using accelerometers can be classified in two main categories: (1) physical-based, and (2) regression-based. Physical-based approaches typically rely on a model of the human body; where velocity or position information is estimated from accelerometer data and kinetic motion and/or segmental body mass is used for to estimating energy expenditure. Regression-based approaches, on the other hand, generally estimate energy expenditure by directly mapping accelerometer data to energy expenditure. Advantageously, regression approaches do not usually require a model of the human body.

One regression-based approach is estimating energy expenditure from a single accelerometer placed at the hip using linear regression. This approach has been extended to using non-linear regression models (i.e., to fully capture the complex relationship between acceleration and energy expenditure) and multiple accelerometers (i.e., to account for upper or lower body motion which is hard to capture from a single accelerometer placed at the hip). Combining accelerometers with other types of sensors, such as heart rate monitors, can improve energy expenditure estimation.

Traditionally, energy expenditure is estimated over sliding windows of one minute length using the number of acceleration counts per minute (e.g., sum of the absolute values of the acceleration signal). Using shorter window lengths and more powerful features (e.g., coefficient of variation, inter-quartile interval, power spectral density over particular frequencies, kurtosis, and skew) can provide more accurate energy expenditure estimates. Moreover, incorporating features based on demographic data (e.g., age, gender, height, and weight) can compensate for inter-individual variations.

A limitation of using accelerometers is in their inability to capture total activity, as accelerometers typically only selectively record movement of the part of the body to which they are attached. Accelerometers worn on the hip are primarily suitable for gait or step approximation, but will not capture upper body movement; if worn on the wrist, locomotion is not accurately recorded. Increasing the number of accelerometers increases accuracy of capturing total body movement but is often not practical due to cost and user discomfort. A more robust measure of total body movement as a proxy for energy expenditure is overall dynamic body exertion (OBDA); this derivation accounts for dynamic acceleration about an organism's center of mass as a result of the movement of body parts, via measurement of orthogonal-axis oriented accelerometry and multiple regression. This approach, for example using using two triaxial accelerometers (one stably oriented in accordance with the main body axes of surge, heave and sway with the other set at a 30-degree offset), has approximated energy expenditure/oxygen consumption more accurately than single-unit accelerometers, but generally requires custom-made mounting blocks in order to properly orient the expensive triaxial accelerometers.

In a specific example, the system and method of the present disclosure are implemented using a commercially available 3D camera (Microsoft's Kinect) and regression algorithms to provide more accurate and robust algorithms for estimating energy expenditure. The Kinect is used to track the movement of a large number (such as 20) of joints of the human body in 3D in a non-intrusive way. This approach can have a much higher spatial resolution than accelerometer based approaches. An additional benefit is also an increase in temporal resolution. Accelerometers typically sample with 32 Hz but they are limited in reporting data in 15 second epochs, whereas the Kinect can report 3D skeletal joint locations with 200 Hz, which allows for real-time estimation of energy expenditure. Benefit of the disclosed approach are that it is non-intrusive, as the user does not have to wear any sensors and its significantly lower cost. For example, the popular Actical accelerometer costs $450 per unit where the Kinect sensor retails for $150.

Kinect is an active vision system designed to allow users to interact with the Xbox 360 video game platform without the need for a hand-held controller. The system uses an infrared camera to detect a speckle pattern projected onto the user's skin in the sensors field of view. A 3D map of the user's body is then created by measuring deformations in the reference speckle pattern. A color camera provides color data to the depth map. Several studies have been performed to assess the accuracy of Kinect by comparing it with some very expensive and highly accurate 3D motion capture systems such as Vicon. The random error of depth measurement increases with increasing distance to the sensor, and ranges from a few millimeters up to about 4 cm at the maximum range of the sensor (i.e., 5.0 m distance from sensor). The Kinect was able to estimate quite accurately the 3D relative positions of four 0.10 cm cubes placed at different distances.

The human body is an articulated system of rigid segments connected by joints. In one implementation, the present disclosure estimates energy expenditure from the continuous evolution of the spatial configuration of these segments. A method to quickly and accurately estimate 3D positions of skeletal joints from a single depth image from Kinect has is described in Shotton, et al., “Real-Time Human Pose Recognition in Parts from Single Depth Images” 2011 IEEE Conference on Computer Vision and Pattern Recognition (CVPR) Jun. 20-25, 2011, 1297-1304 (June 2011), incorporated by reference herein. The method provides accurate estimation of twenty 3D skeletal joint locations at 200 frames per second and is invariant to pose, body shape, clothing, etc. The skeletal joints include hip center, spine, shoulder center, head, L/R shoulder, L/R elbow, L/R wrist, L/R hand, L/R hip, L/R knee, L/R ankle, and L/R foot. The estimated joint locations include information about the direction of the person is facing (i.e., can distinguish between the left and right limb joints).

The present disclosures estimates energy expenditure using computing motion-related features from 3D joint locations and mapping them to ground truth energy expenditure using state-of-the-art regression algorithms. In one implementation, ground truth energy expenditure is estimated by computing the mean value over the same time window of energy expenditure data collected using an indirect calorimeter (e.g., in METs). METs are the number of calories expended by an individual while performing an activity in multiples of his/her resting metabolic rate (RMR). METs can be converted to calories by measuring or estimating an individual's RMR.

Having information about 3D joint locations allows acceleration information in each direction to be computed. Thus, the same type of features previously introduced in the literature using accelerometers can be computed using the present disclosure. The present disclosure can provide greater accuracy and at a higher spatial and temporal resolution. The present disclosure can also be used to extract features from powerful, view-invariant, representations schemes of human motion, such as histograms of 3D joints, as described in Xia, et al., “View invariant human action recognition using histograms of 3d joints,” 2nd International Workshop on Human Activity Understanding from 3D Data (HAU3D), in conjunction with IEEE CVPR 2012, Providence, R.I., 2012, incorporated by reference herein (available at cvrc.ece.utexas.edu/Publications/Xia_HAU3D12.pdf).

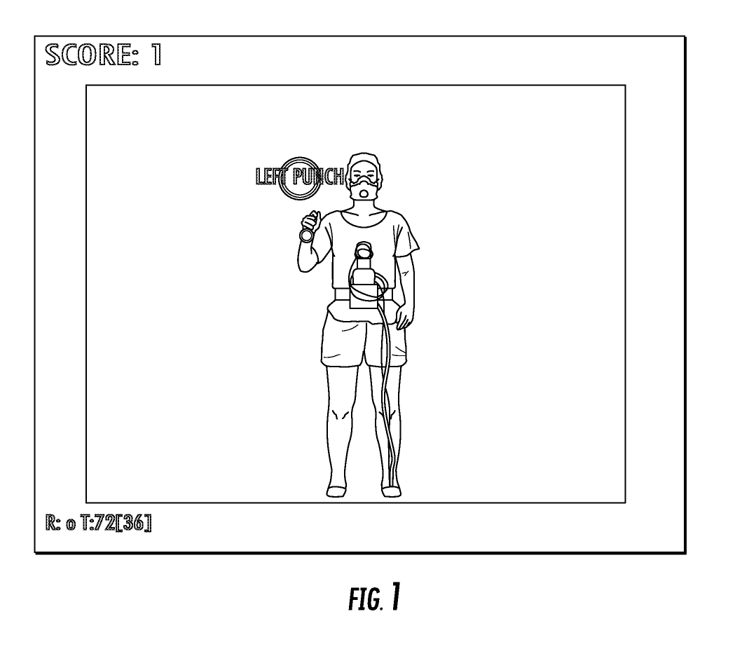

As described in Xia, a spherical coordinate system (see its FIG. 1) is associated with each subject and 3D space is partitioned into n bins. The center of the spherical coordinate system is determined by the subjects hip center while the horizontal reference axis is determined by the vector from the left hip center to the right hip center. The vertical reference axis is determined by the vector passing through the center and being perpendicular to the ground plane. It should be noted that since joint locations contain information about the direction the person is facing, the spherical coordinate system can be determined in a viewpoint invariant way. The histogram of 3D joints is computed by partitioning the 3D space around the subject into n bins. Using the spherical coordinate system ensures that any 3D joint can be localized at a unique bin. To compute the histogram of 3D joints, each joint casts a vote to the bin that contains it. For robustness, weighted votes can be cast to nearby bins using a Gaussian function. To account for temporal information, the technique can be extended by computing histograms of 3D joints over a non-overlapping sliding window. This can be performed by adding together the histograms of 3D joints computed at every frame within the sliding window. Parameters that can be optimized in specific example include (i) the number of bins n, (ii) the parameters of the Gaussian function, and (iii) the length of the sliding window. To obtain a compact set of discriminative features from the histograms of 3D joints, dimensionality reduction will be applied, for example, Regularized Nonparametric Discriminant Analysis. To learn the complex relationship between histograms of 3D joints and energy expenditure we will investigate modern Regression methods such as Online Support Vector Regression, Boosted Support Vector Regression, Gaussian Processes, and Random Regression Forests can be used to associate histograms of 3D joints and energy expenditure, as described in the following references, each of which is incorporated by reference herein in it's entirety, Wang, et al., “Improving target detection by coupling it with tracking,” Mach. Vision Appl. 20(4):205-223 (April 2009); Asthana, et al., “Learning based automatic face annotation for arbitrary poses and expressions from frontal images only,” 2009 IEEE Conference on Computer Vision and Pattern Recognition 1635-1642 (June 2009); Williams, et a., “Sparse and semi-supervised visual mapping with the s3gp,” 2006 IEEE Conference on Computer Vision and Pattern Recognition 1:230-237 (June 2006); Fanelli, et al., “Real time head pose estimation with random regression forests,” 2011 IEEE Conference on Computer Vision and Pattern Recognition (CVPR), 617-624 (June 2011).

The depth accuracy of Kinect has been evaluated for static objects. To test the accuracy of the Kinect for motions, an exergame was developed that involved gross motor skills. This game involved punching and kicking virtual objects that were rendered in front of an image of the user (FIG. 1). The accuracy of the Kinect was measured using an optical 3D motion tracking system that had a tracking accuracy of 1 mm. With markers attached at the wrists (which are joints in the Kinect skeletal model) a number of experiments found a tracking error of the Kinect that was less than 10 mm.

Movements in the skeletal model and be correlated with calorie expenditure data, such as using pulmonary data, by having subjects play a video game that involves gross motor skills (e.g., punches, kicks and jumps). In one example, forty healthy students are recruited to participate in the measurement. In order to get a robust dataset, the participants are chosen with a variety of genders, ethnicities, and body types, such a as defined by body mass index (BMI). Subjects height and weight data are recorded. During this initial recruitment visit, subjects additionally undergo body composition assessment using dual-energy X-ray absorptiometry in order to facilitate classification according to percent fat, bone, and lean muscle mass. Pulmonary data is collected using a Cosmed K4b2 portable telemetric breath-by-breath gas analysis system, utilizing an 18 mm turbine. The system is calibrated before each game trial in the following way: (a) the turbine is calibrated for volumetric flow using a 3.0 L calibrated gas syringe, and (b) the CO₂ and O₂ sensors are calibrated with a standard gas mixture of 16% O₂:5% CO₂. Subject body composition is assessed using the GE Lunar dual-energy x-ray absorptiometer. Each well-hydrated subject is analyzed for lean mass (as skeletal muscle mass), fat mass, and bone mineral content.

Subjects are shown how to play the game, which involves punching and kicking targets that are indicated using visual cues (FIG. 1). Targets are defined in front of the user at arm's length to stimulate the largest amount of physical activity. Users jump over targets to stimulate whole body movements. Prior to playing the game the game calibrates itself to the user's height and reach of their limbs to enable the user to play the game in place, which is typical for exergames. The user has a limited amount of time to destroy each target, which is indicated by having the target change color from green to yellow to red. Users score points for each target destroyed and the faster targets are destroyed the more points a user scores, which is a motivating factor. Initially the game only includes upper body motions, but becomes progressively harder to play, e.g. upper and lower motions have to be performed simultaneously and the user also has to jump. Targets also appear faster and farther away from the user. The motivation behind this is to stimulate larger amounts of physical activity. After familiarizing themselves with the game play the users are equipped with the pulmonary gas exchange system, which continuously collects pulmonary gas exchange data throughout the exercise bout. Data is collected in 1 minute blocks with one minute rest between each level.

The increased spatial and temporal resolution of being able to track skeletal joints motions will improve the accuracy in estimating energy expenditure compared with accelerometer based approaches. Data collected from a homogenous population of males with both (1) BMI<25 and (2) body fat percentage less than 17.5%, in order to minimize potential inter-individual variation in energy expenditure due to physiological differences such as gender and gross phenotype; better allows for the exploration of features that are most useful in predicting energy expenditure. The collected data is partitioned in training and test data. The parameters of the regression model are estimated using the training data while the test data is used for assessing the performance of the disclosed approach. Three different regression models are trained. Two regression models simulate accelerometer based approaches with features based on acceleration data with a spatial resolution of three joints (wrist, hip, leg) and five joints (wrist, hip, legs). Acceleration data is computed from observed movement data from the respective joints, the relative limited sensitivity of accelerometers (0.05 to 2 G) and temporal resolution (15 second epochs) are further modeled. A third regression model uses features computed from joint movements from all 20 skeletal joints. If desired, joints can be identified which provide the most important information. For example, some joints, such as hand/wrist and ankle/foot, are very close to each other; so they may contain redundant information. Similar, because some specific joints (shoulder, elbow and wrist) are connected, redundant information may be present. If so, features can be defined at a higher level of abstraction, i.e, limbs. Whether to use a higher level of abstraction (less granular data) can also depend on the desired balance between processing speed/load and accuracy in measuring energy expenditure. Features from view-invariant, representations schemes of human motion, such as histograms of 3D joints, can be used in addition to or in place of more standard features, e.g., acceleration and velocity. Data analysis can be subject dependent or subject independent. For subject independent evaluation, a one-left-out approach can be used. That is, training will be performed using the data of all the subjects but one and tested the performance on the left-out subject. This procedure is repeated for all the subjects and the results averaged. For subject dependent evaluation, a k-fold cross-validation approach can be used.

Subject independent energy expenditure estimation is typically more difficult than subject dependent estimation, as commonly employed regression models fail to account for physiological differences between subject ‘sets’ utilized for model training/validation and individual subjects testing with that model. In order to increase the applicability of regression models to varied subject phenotypes, additional data is collected from thirty participants, including 10 males with BMI over 25 and percentage body fat over 17.5%, and 20 females, including 10 with a BMI<25 and percent body fat under 23.5%, and 10 with BMI>25 and percent body fat over 23.5%. This data is combined from the previously obtained data in order to create a more robust data set, as there may be significant inter-individual differences in energy expenditure due to differences in gender and body type. New features can be defined to capture such differences. The subject population can be stratified according to body composition. Features that calculate distances between joints as a supplemental, morphometric descriptor of phenotype can be included. Regression models that can be used include regression ensembles, an effective technique in machine learning for reducing generalization error by combining a diverse population of regression models.

The type of activity a user engages in can have a significant effect on energy expenditure. The exergame used to collect training data may only include gross motor motions, such as punches, kicks and jumps. Previous work on energy expenditure classified activities in different types and employing a different regression model to estimate energy expenditure for each activity type. This classifying all types of possible activities can be a difficult or limit the applicability of the measurement. According to the present disclosure, robust features are defined that are independent of the type of the activity. To help identify this features, in addition to collecting pulmonary gas exchange data of subjects using an exergame, subjects will either play a: (1) tennis based exergame or (2) a ball punching and kicking game. For example, existing exergames for the Kinect using an additional Kinect sensor for input whose location will be calibrated with the Kinect sensor for capturing joint data.

Skeletal joint positions and pulmonary gas exchange data is collected while the user is playing the game. The subjects' height and weight is collected. Qualitative experiences are acquired using a questionnaire to assess the non-intrusiveness of the disclosed technique.

### Example

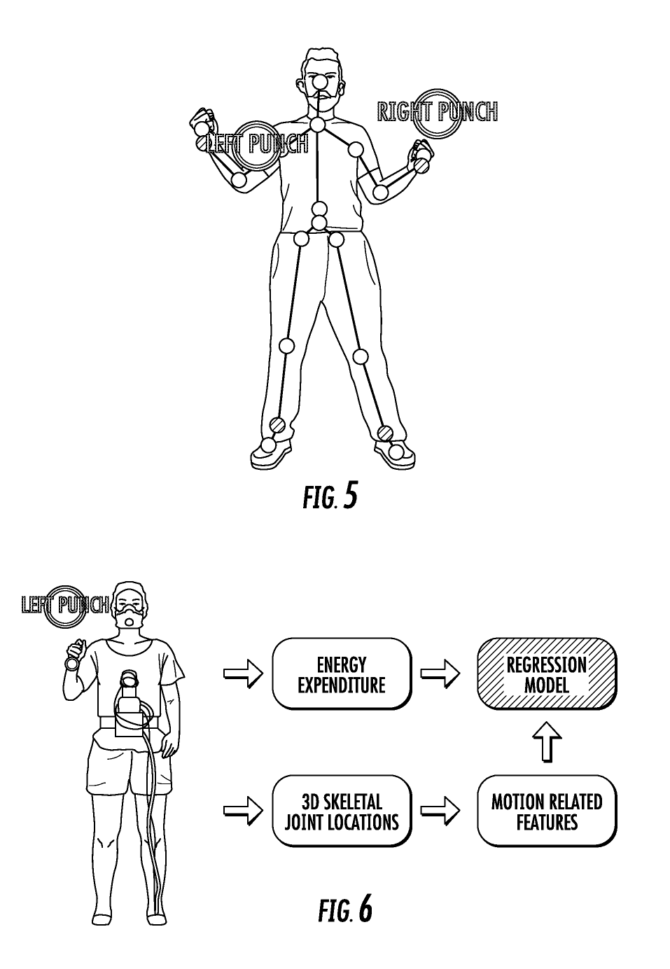

The present disclosure provides a non-calorimetric technique that can predict EE of exergaming activities using the rich amount of kinematic information acquired using 3D cameras, such as commercially available 3D cameras (Kinect). Kinect is a controllerless input device used for playing video games and exercise games for the Xbox 360 platform. This sensor can track up to six humans in an area of 6 m² by projecting a speckle pattern onto the users body using an IR laser projector. A 3D map of the users body is then created in real-time by measuring deformations in the reference speckle pattern. A single depth image allows for extracting the 3D position of 20 skeletal joints at 200 frames per second. This method is invariant to pose, body shape and clothing. The joints include hip center, spine, shoulder center, head, shoulder, elbow, wrist, hand, hip, knee, ankle, and foot (See FIG. 5). The estimated joint locations include the direction that the person is facing, which allows for distinguishing between the left and right joints for shoulder, elbow, wrist, hand, hip, knee, ankle and foot. Studies have investigated the accuracy of Kinect, which found that the depth measurement error ranges from a few millimeters at the minimum range (70 cm) up to about 4 cm at the maximum range of the sensor (6.0 m).

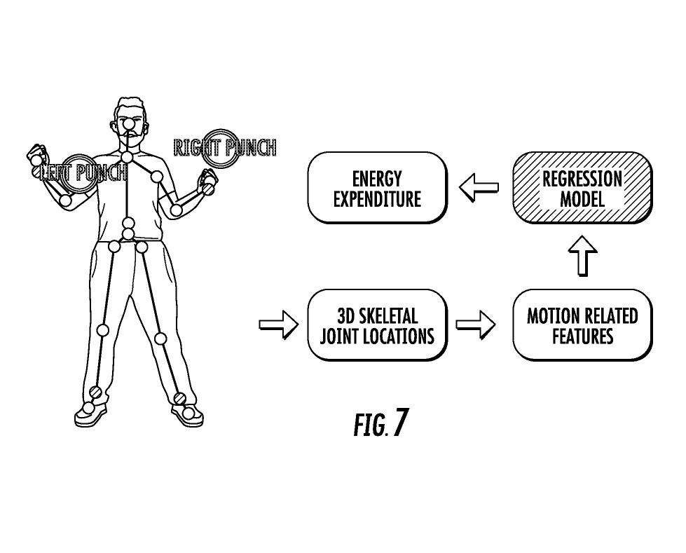

In a specific implementation, the disclosed technique uses a regression based approach by directly mapping kinematic data collected using the Kinect to EE, since this has shown good results without requiring a model of the human body. The EE of playing an exergame is acquired using a portable VO2 metabolic system, which provides the ground truth for training a regression model (see FIG. 6). Given a reasonable amount of training data, the regression model can then predict EE of exergaming activities based on kinematic data captured using a Kinect sensor (see FIG. 7). Accelerometer based approaches typically estimate EE using a linear regression model over a sliding window of one-minute length using the number of acceleration counts per minute (e.g., the sum of the absolute values of the acceleration). A recent study found several limitations for linear regression models to accurately predict EE using accelerometers. Nonlinear regression models may be able to better predict EE associated with upper body mo-tions and high-intensity activities.

In one implementation of the disclosed technique, Support Vector Regression (SVR) is used, a popular regression technique that has good generalizability and robustness against outliers and supports non-linear regression models. SVR can approximate complex non-linear relationships using kernel transformations. Kinect allows for recording human motion at a much higher spatial and temporal resolution. Where accelerometer based approaches are limited to using up to five accelerometers simultaneously, the disclosed technique can take advantage of having location information of 20 joints. This allows for detecting motions of body parts that do not have attached accelerometers such as the elbow or the head. Though accelerometers sample at 32 Hz, they report accumulated acceleration data in 1 second epochs. Their sensitivity is also limited (0.05 to 2 G). Because the disclosed technique acquires 3D joint locations at 200 Hz, accelerations can be calculated more accurately and with a higher frequency. Besides using acceleration, features from more powerful, view-invariant, spatial representation schemes of human motion can be used, such as histograms of 3D joints. Besides more accurate EE assessment, the disclosed technique has a number of other benefits: (1) Accelerometers can only be read out using an external reader, where the disclosed technique can predict EE in real time, which may allow for real-time adjustment of the intensity of an exergame; (2) Subjects are not required to wear any sensors, though they must stay within range of the Kinect sensor; and (3) Accelerometers typically cost several hundreds of dollars per unit whereas a Kinect sensor retails for $150.

An experiment was conducted to demonstrate the feasibility of the disclosed method and system to accurately predict the energy expenditure (EE) of playing an exergame. This experiment provides insight into the following two re-search questions: (1) What type of features are most useful in predicting EE? (2) What is the accuracy compared with accelerometer based approaches?

Instrumentation

For the experiment, the Kinect for Windows sensor was used, which offers improved skeletal tracking over the Kinect for Xbox 360 sensor. Though studies have investigated the accuracy of Kinect, these were limited to non-moving objects. The accuracy of the Kinect to track moving joints was measured using an optical 3D motion tracking system with a tracking accuracy of 1 mm. The arms were anticipated to be the most difficult portion of the body to track, due to their size; therefore, a marker was attached at the wrist of subjects, close to wrist joints in the Kinect skeletal model. A number of preliminary experiments with two subjects performing various motions with their arms found an average tracking error of less than 10 mm, which was deemed acceptable for our experiments. EE was collected using a Cosmed K4b2 portable gas analysis system, which measured pulmonary gas exchange with an accuracy of ±0.02% (O2), ±0.01% (CO2) and has a response time of 120 ms. This sys-tem reports EE in Metabolic Equivalent of Task (MET); a physiological measure expressing the energy cost of physical activities. METs can be converted to calories by measuring an individual's resting metabolic rate.

An exergame was developed using the Kinect SDK 1.5 and which involves destroying virtual targets rendered in front of an image of the player using whole body gestures (See FIG. 1 for a screenshot). This game is modeled after popular exergames, such as EyeToy:Kinetic and Kinect Adventures. A recent criticism of exergames is that they only engage their players in light and not vigorous levels of physical activity, where moderate-to-vigorous levels of physical activity are required daily to maintain adequate health and fitness. To allow the method/system of the present disclosure to distinguish between light and vigorous exergames, a light and a vigorous mode was implemented in the game of this example. The intensity level of any physical activity is considered vigorous if it is greater than 6 METs and light if it is below 3 METs. Using the light mode, players destroy targets using upper body gestures, such as punches, but also using head-butts. Gestures with the head were included, as this type of motion is difficult to measure using accelerometers, as they are typically only attached to each limb. This version was play tested with the portable metabolic system using a number of subjects to verify that the average amount of EE was below 3 METs. For the vigorous mode, destroying targets using kicks were added, as previous studies show that exergames involving whole body gesture stimulate larger amounts of EE than exergames that only involve upper body gestures. After extensive play testing, jumps were added to assure the average amount of EE of this mode was over 6 METs.

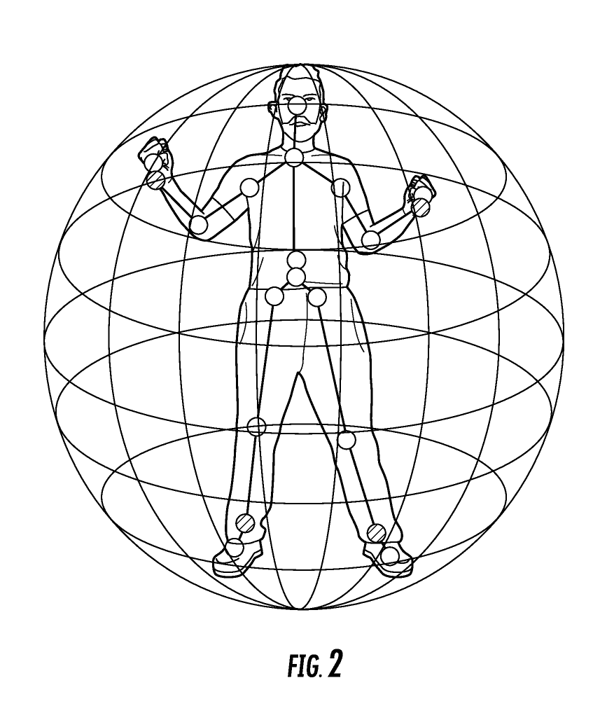

A target is first rendered using a green circle with a radius of 50 pixels. The target stays green for 1 second before turning yellow and then disappears after 1 second. The player scores 5 points if the target is destroyed when green and 1 when yellow as to motivate players to destroy targets as fast as possible. A jump target is rendered as a green line. A sound is played when each target is successfully destroyed. For collision detection, each target can only be destroyed by one specific joint (e.g., wrists, ankles, head). A text is displayed indicating how each target needs to be destroyed, e.g., “Left Punch” (see FIG. 2).

An initial calibration phase determines the length and posi-tion of the player's arms. Targets for the kicks and punches are generated at an arm's length distance from the player to stimulate the largest amount of physical activity without having the player move from their position in front of the sensor. Targets for the punches are generated at arm's length at the height of the shoulder joints with a random offset in the XY plane. Targets for the head-butts are generated at the distance of the player's elbows from their shoulders at the height of the head. Jumps are indicated using a yellow line where the players have to jump 25% of the distance between the ankle and the knee. Up to two targets are generated every 2 seconds. The sequence of targets in each mode is generated pseudo-randomly with some fixed probabilities for light (left punch: 36%, right punch: 36%, two punches: 18%, head-butt: 10%) and for the vigorous mode (kick: 27%, jump: 41%, punch: 18%, kick+punch: 8%, head-butt: 5%). Targets are generated such that the same target is not selected sequentially. All variables were determined through extensive play testing as to assure the desired METs were achieved for each mode. While playing the game the Kinect records the subject's 20 joint positions in a log file every 50 milliseconds.

Participants

Previous work on EE estimation has shown that subject independent EE estimation is more difficult than subject dependent estimation. This is because commonly employed regression models fail to account for physiological differences between subject data used to train and test the regression model. For this example, the primary interest is in identifying those features that are most useful in predicting EE. EE will vary due to physiological features, such as gender and gross phenotype. To minimize potential inter-individual variation in EE, which helps focus on identifying those features most useful in predicting EE; data was collected from a homogeneous healthy group of subjects. The following criteria were used: (1) male; (2) body mass index less than 25; (3) body fat percentage less than 17.5%; (4) age between 18 and 25; (5) exercise at least three times a week for 1 hour. Subjects were recruited through flyers at the local campus sports facilities. Prior to participation, subjects were asked to fill in a health questionnaire to screen out any subjects who met the inclusion criteria but for whom we anticipated a greater risk to participate in the trial due to cardiac conditions or high blood pressure. During the intake, subjects' height, weight and body fat were measured using standard anthropomorphic techniques to assure subjects met the inclusion criteria. Fat percentage was acquired using a body fat scale. A total of 9 males were recruited (average age 20.7 (SD=2.24), weight 74.2 kg (SD=9.81), BMI 23.70 (SD=1.14), fat % 14.41 (SD=1.93)). The number of subjects in this Example is comparable with related regression based studies. Subjects were paid $20 to participate.

Data Collection

User studies took place in an exercise lab. Subjects were asked to bring and wear exercise clothing during the trial. Before each trial the portable VO2 metabolic system was calibrated for volumetric flow using a 3.0 L calibrated gas syringe, and the CO2 and O2 sensors were calibrated using a standard gas mixture of 02:16% and CO2:5% according to the manufacturer's instructions. Subjects were equipped with the portable metabolic system, which they wore using a belt around their waist. Also they were equipped with a mask using a head strap where we ensured the mask fit tightly and no air leaked out. Subjects were also equipped with five Actical accelerometers: one on each wrist, ankle and hip to allow for a comparison between techniques. Prior to each trial, accelerometers were calibrated using the subject's height, weight and age. It was assured there was no occlusion and that subjects were placed at the recommended distance (2 m) from the Kinect sensor. Subjects were instructed what the goal of the game was, i.e., score as many points as possible within the time frame by hitting targets as fast as possible using the right gesture for each target. For each trial, subjects would first play the light mode of the game for 10 minutes. Subjects then rested for 10 minutes upon which they would play the vigorous mode for 10 minutes. This order minimizes any interference effects, e.g., the light bout didn't exert subjects to such an extent that it is detrimental to their performance for the vigorous bout. Data collection was limited to ten minutes, as exergaming activities were considered to be anaerobic and this Example was not focused on predicting aerobic activities.

Training the Regression Model

Separate regression models were trained for light and vigorous activities as to predict METs, though all data is used to train a single classifier for classifying physical activities. Eventually when more data is collected, a single regression model can be trained, but for now, the collected data represents disjunct data sets. An SVM classifier was used to classify an exergaming activity into being light or vigorous; only kinematic data and EE for such types of activities was collected. Classifier and regression models were implemented using the LibSVM library. Using the collected ground truth, different regression models were trained so as to identify which features or combinations of features yield the best performance. Using the skeletal joint data obtained, two different types of motion-related features are extracted: (1) Acceleration of skeletal joints; and (2) Spatial information of skeletal joints.

Acceleration: acceleration information of skeletal joints is used to predict the physical intensity of playing exergames. From the obtained displacement data of skeletal joints, the individual joint's acceleration is calculated in 50 ms blocks, which is then averaged over one-minute intervals. Data was partitioned in one-minute blocks to allow for comparison with the METs predicted by the accelerometers. Though the Kinect sensor and the Cosmed portable metabolic system can sample with a much higher frequency, using smaller time windows won't allow for suppressing the noise, which exists in the sampled data. There is a significant amount of corre-lation between accelerations of joints (e.g., when the hand joint moves, the wrist and elbow often move as well as they are linked). To avoid over-fitting the regression model, the redundancy in the kinematic data was reduced using Principal Component Analysis (PCA) where five acceleration features were selected that preserve 90% of the information for the light and 92% for the vigorous model. PCA was applied because the vectors were very large and it was desired to optimize the performance of training the SVR. It was verified experimentally that applying PCA did not affect prediction performance significantly.

Spatial: to use joint locations as a feature, a view-invariant representation scheme was employed called joint location binning. Unlike acceleration, joint binning can capture specific gestures, but it cannot discriminate between vigorous and less vigorous gestures. As acceleration already captures this, joint binning was evaluated as a complementary feature to improve performance. Joint binning works as follows: 3D space was partitioned in n bins using a spherical coordinate system with an azimuth (θ) and a polar angle (φ) that was centered at the subject's hip and surrounds the subject's skeletal model (see FIG. 2). The parameters for partitioning the sphere and the number of bins that yielded the best performance for each regression model were determined experimentally. For light, the best performance was achieved using 36 bins where θ and φ were partitioned into 6 bins each. For vigorous, 36 bins were used where θ was partitioned into 12 bins and φ into 3 bins. Binning information for each joint was managed by a histogram with 36 bins; with a total of 20 histograms for all joints were used as a feature vector. Histograms of bin frequencies were created by mapping the 20 joints to appropriate bin locations over one-minute time interval with a 50 ms sampling rate. When bin frequencies are added, the selected bin and its neighbors get votes weighted linearly based on the distance of the joint to the center of the bin it is in. To reduce data redundancy and to extract dominant features from the 20 histograms, PCA was used to extract five features retaining 86% of information for light and 92% for the vigorous activities. As the subject starts playing the exergame, it takes some time for their metabolism and heart rate to increase; therefore the first minute of collected data is excluded from our regression model. A leave-one-out approach was used to test the regression models, where data from eight subjects was used for training and the remaining one for testing. This process was repeated so that each subject was used once to test the regression model.

Results

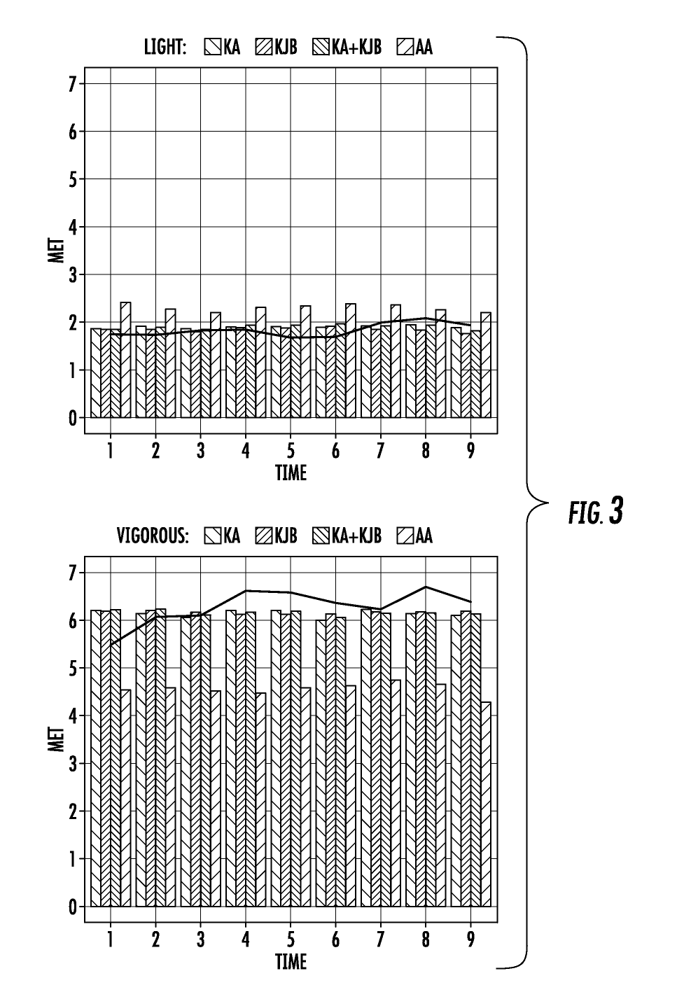

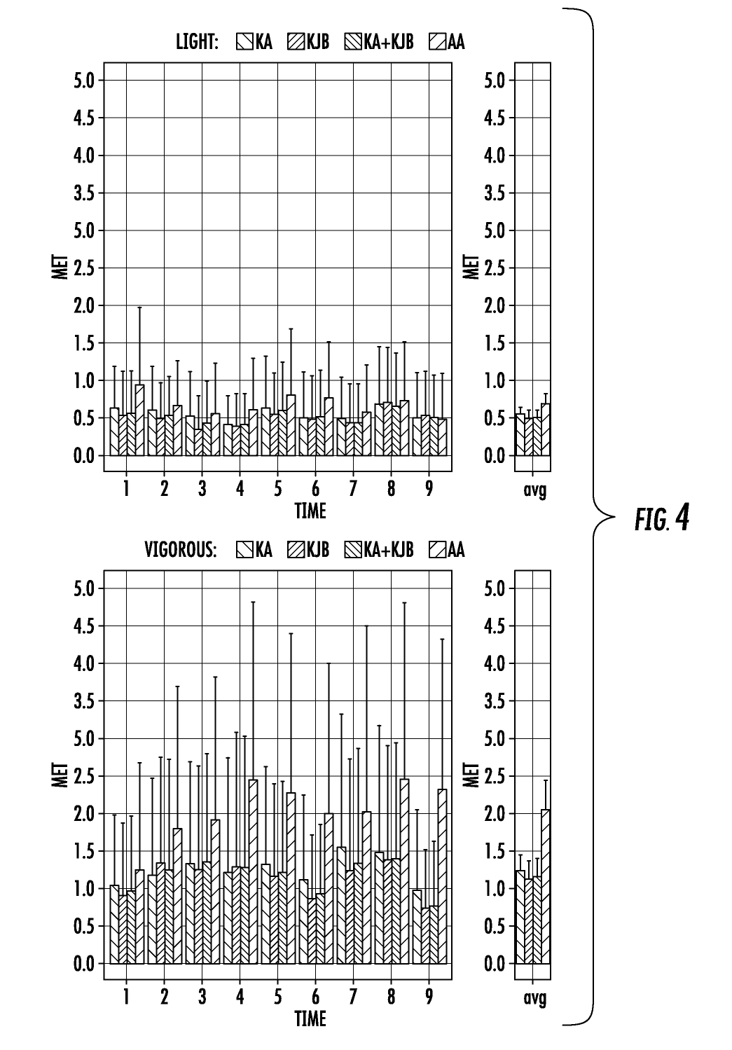

FIG. 3 shows the predicted METs of the light and vigorous regression models using three sets of features: (1) acceleration (KA); (2) joint position (KJB) and (3) both (KA+KJB). For the accelerometers (AA), METs are calculated by averaging the METs of each one of the five accelerometers used according to manufacturer's specifications. METs are predicted for each subject and then averaged over the nine subjects; METs are reported in one-minute increments. On average the METs predicted by the regression models are within 17% of the ground truth for light and within 7% for vigorous, where accelerometers overestimate METs with 24% for the light and underestimate METs with 28% for vigorous. These results confirm the assumption that accelerometers predict EE of exergames poorly. The root mean square (RMS) error as a measure of accuracy was calculated for each technique (see FIG. 4). A significant variance in RMS error between subjects can be observed due to physiological differences between subjects. Because the intensity for each exergame is the same throughout the trial, METs were averaged over the nine-minute trial and performance of all techniques were compared using RMS. For the light exergame, a repeated-measures ANOVA with a Greenhouse-Geisser correction found no statistically significant difference in RMS between any of the techniques (F_{1.314,10.511}=3.173, p=0.097). For the vigorous ex-ergame, using the same ANOVA, a statistically significant difference was found (F_{1.256,10.044}=23.964, p<0.05, partial η²=0.750). Post-hoc analysis with a Bonferroni adjustment revealed a statistically significant difference between MET predicted by all regression techniques and the accelerometers (p<0.05). Between the regression models, no significant difference in RMS between the different feature sets was found (p=0.011).

Classifying Exergame Intensity

To be able to answer the question whether an exergame en-gages a player into light or vigorous physical activity, an SVM was trained using all the data collected in our experiment. A total of 162 data points were used for training and testing with each data point containing one-minute of averaged accelerations for each of the 20 joints. Using 9-fold cross-validation an accuracy of 100% was achieved. Once an activity was classified, the corresponding regression model could be used to accurately predict the associated METs.

For vigorous exergaming activities the method/system of the present disclosure predicts MET more accurately than accelerometer-based approaches. This increase in accuracy may be explained by an increase in spatial resolution that allows for capturing gestures, such as head-butts more accurately, and the ability to calculate features more precisely due to a higher sampling frequency. The increase in performance should be put in context, however, as the regression model was trained and tested using a restricted set of gestures, where accelerometers are trained to predict MET for a wide range of motions, which inherently decreases their accuracy.

It was anticipated that joint binning would outperform joint acceleration, as it allows for better capturing of specific gestures; but the data showed no significant difference in RMS error between both features and their combination. Joint binning however, may yield a better performance for exergames that include more sophisticated sequences of gestures, such as sports based exergames. A drawback of using joint binning as a feature is that it restricts predicting MET to a limited set of motions that were used to train the regression model. The histogram for joint binning for an exergame containing only upward punches looks significantly different from the same game that only contains forward punches. The acceleration features for both gestures, however, are very similar. If it can be assumed that their associated EE do not differ significantly, acceleration may be a more robust feature to use, as it will allow for predicting MET for a wide range of similar gestures that only vary in the direction they are performed, with far fewer training examples required than when using joint binning. Because SVM uses acceleration as a feature, it may already be able to classify the intensity of exergames, who use different gestures from the one used in this experiment.

The exergame used for training the regression model used a range of different motions, but it doesn't cover the gamut of gestures typically used in all types of exergames, which vary from emulating sports to dance games with complex step patterns. Also, the intensity of the exergame for training the regression models in this example was limited to two extremes, light and vigorous, as these are considered criteria for evaluating the health benefits of an exergame. Rather than having to classify an exergame's intensity a priori, a single regression model that can predict MET for all levels of intensity would be more desirable, especially since moderate levels of physical activity are also considered to yield health benefits.

Though no difference was found in performance between acceleration and joint position, there are techniques to refine these features. For example, acceleration can be refined by using coefficient of variation, inter-quartile intervals, power spectral density over particular frequencies, kurtosis, and skew. Joint binning can be refined by weighing bins based on the height of the bin or weighing individual joints based on the size of the limb they are attached to. Since the emphasis of this Example was on identifying a set of features that would allow us to predict energy expenditure, comparisons were not performed using different regression models. Different regression models can be used, such as random forests regressors, which are used by the Kinect and which typically outperform SVR's for relatively low dimensionality problems spaces like those in this Example.

A high variance in RMS error between subjects was observed despite efforts to minimize variation in EE by drawing subjects from a homogeneous population. Demographic data should be considered to train different regression models to compensate for inter-individual variations. Alternatively the regression result could be calibrated by incorporating demographic information as input to the regression model or correcting the regression estimates to compensate for demographic differences. Since exergames have been advocated as a promising health intervention technique to fight childhood obesity, it is important to collect data from children. There is an opportunity to use the Kinect to automatically identify demographic data, such as gender, age, height and weight, and automatically associate a regression model with it, without subjects having to provide this information in advance. It may be advantageous to interpolate between regression models in the case that no demographic match can be found for the subject.

It is to be understood that the above discussion provides a detailed description of various embodiments. The above descriptions will enable those skilled in the art to make many departures from the particular examples described above to provide apparatuses constructed in accordance with the present disclosure. The embodiments are illustrative, and not intended to limit the scope of the present disclosure. The scope of the present disclosure is rather to be determined by the scope of the claims as issued and equivalents thereto.

1. A computer implemented method of determining energy expenditure associated with a user's movement comprising:
obtaining a plurality of video images of a subject;
from the plurality of video images, determining a first location of a first joint of the subject at a first time;
from the plurality of video images, determining a second location of the first joint of the subject at a second time;
associating the movement of the first joint of the subject between the first and second location with an energy associated with the movement.

2. The computer implemented method of claim 1, wherein associating the movement of the first joint of the subject between the first and second location with an energy associated with the movement comprises using a regression model.

3. The computer implemented method of claim 1, wherein associating the movement of the first joint of the subject between the first and second location with an energy associated with the movement comprises using a view-invariant representation scheme of motion.

4. The computer implemented method of claim 3, wherein the view-invariant representation scheme of motion comprising a histogram of 3D joints.

5. The computer implemented method of claim 1, wherein associating the movement of the first joint of the subject between the first and second location with an energy associated with the movement comprises associating the movement with a library of motions and their associated energy expenditures.

6. The computer implemented method of claim 5, wherein the library comprises energy expenditure data based on pulmonary data.

7. The computer implemented method of claim 1, further comprising calculating the distance between the first joint of the subject and a second joint of the subject.

8. The computer implemented method of claim 7, wherein determining the location of the first and second location of the first joint comprises associating the first and second joints as a first combined features and determining a first location of the combined feature at the first time and a second location of the combined feature at a second time.

9. The computer implemented method of claim 8, further comprising calculating the distance between the first joint and the second joint.

10. The computer implemented method of claim 9, wherein the distance between the first and second joint is used as a morphometric descriptor of phenotype.

11. The computer implemented method of claim 8, wherein the combined feature represents a limb.

12. The computer implemented method of claim 8, wherein the combined feature represents at least a portion of a limb.

13. The computer implemented method of claim 1, further comprising, from the plurality of video images, determining a first location of a second joint of the subject at a first time;
from the plurality of video images, determining a second location of the second joint of the subject at a second time;
associating the movement of the second joint of the subject between the first and second location with an energy associated with the movement.

14. The computer implemented method of claim 1, further comprising, from the plurality of video images, determining a first location of a plurality of joints of the subject at a first time, the first joint being one of the plurality of joints;
from the plurality of video images, determining a second location of the each of the plurality of joints of the subject at a second time;
associating the movement of each of the plurality of joints of the subject between the first and second location with an energy associated with the movement.

15. The computer implemented method of claim 14, wherein the plurality of joints comprises at least five joints.

16. The computer implemented method of claim 14, wherein the plurality of joints comprises at least ten joints.

17. The computer implemented method of claim 14, wherein the plurality of joints comprises at least twenty joints.

18. The computer implemented method of claim 14, further comprising calculating an interjoint relationship between the first joint and a second joint of the plurality of joints.

19. The computer implemented method of claim 1, wherein the energy is an estimated energy.

20. The computer implemented method of claim 1, further comprising calculating an acceleration of the first joint of the subject as the first joint moves between the first and second positions.

21-25. (canceled)
